(19) United States
(12) Patent Application Publication
(10) Pub. No.: US 2006/0083708 A1
(43) Pub. Date: Apr. 20, 2006

(54) Composition using mineral salts for cosmetic or therapeutic treatment

(76) Inventor: Sam Schwartz, Woodland Hills, CA (US)

Correspondence Address: GREENBERG TRAURIG LLP, 2450 COLORADO AVENUE, SUITE 400E, SANTA MONICA, CA, 90404, US

(21) Appl. No.: 11/218,939

(22) Filed: Sep. 1, 2005

Related U.S. Application Data
(63) Continuation-in-part of application No. 09/220,986, filed on Dec. 23, 1998.
(60) Provisional application No. 60/072,477, filed on Jan. 26, 1998.
(60) Provisional application No. 60/606,641, filed on Sep. 1, 2004.

(30) Foreign Application Priority Data
Aug. 2, 2000 (EP) 0116679.2

(51) Int. Cl. A61K 8/97; A61K 33/14; A61K 36/288; A61K 36/61

(52) U.S. Cl. 424/74; 424/677; 424/679; 424/680; 424/769; 424/764; 424/729; 424/745; 424/728

(57) ABSTRACT

A cosmetic and tissue cleansing composition including mineral salts, preferably Dead Sea Mineral Salts, Tea Tree Extract and Urea as an effective agent to minimize the effect of itching on the skin from a condition caused by psoriasis. In another form, the composition can be used as an exfoliating or scrubbing composition. The composition being formulated with a non-oily base which is water soluble such that the granules which are mineral salts act as exfoliating or scrubbing agents. The composition can also be formed for dental or mouth use. The composition can be used for therapeutic uses together with organic herbs and selectively aromatic essences.

### CROSS-REFERENCE TO RELATED APPLICATIONS

This application is a continuation-in-part and claims priority to U.S. Utility Application Ser. No. 09/220,986, filed Dec. 23, 1998, and entitled “COSMETIC AND TISSUE CLEANSING AND MOISTURIZING COMPOSITION”, which claims priority of U.S. Provisional Application Serial No. 60/072,477, filed Jan. 26, 1998, and entitled “COSMETIC COMPOSITION”. This application claims also the benefit of priority to U.S. Provisional Application Serial No. 60/606,641, filed on Sep. 1, 2004 and entitled “COMPOSITION USING MINERAL SALTS FOR THERAPEUTIC TREATMENT”, all of which incorporated by reference in the present application in their entirety.

The present application also relates to European Patent Application Number 0116679.2, filed Aug. 2, 2000, published on Jul. 2, 2001 as EP 1 074 245 A2 ; and U.S. Provisional Application Serial No. 60/147,634, filed Aug. 6, 1999, and entitled “COMPOSITION FOR USING MINERAL SALTS FOR THERAPEUTIC TREATMENT”, the contents of which are incorporated by reference herein.

### FIELD OF THE DISCLOSURE

This application is directed to the field of cosmetic, therapeutic and cleansing compositions.

### BACKGROUND OF THE DISCLOSURE

Modern environmental conditions, such as heating and air conditioning, dryness, exposure to the sun, and is environment pollution exert severe stress on the skin and accelerate the skin deterioration.

This can result in wrinkles, loss of firmness and elasticity, discoloration, dryness and other cosmetically undesirable effects. Additionally, medical conditions, such as psoriasis, can cause skin conditions which are exhibited as skin flaking and loss of skin tissue.

Although a number of cosmetic, cleansing and moisturizing compositions for use on the skin already exist, it is desirable to provide a cosmetic, cleansing and moisturizing composition for skin treatment, which can provide advantages over those which are currently known.

It is also desirable to have compositions for use not only on humans but also on animals, as well as compositions that can be used within the mouth as a cleanser of the mouth or the teeth.

Different compositions are known using mineral salts from the Dead Sea, for instance, as bath salts.

It is not, however, known to use such minerals for other cosmetic or cleansing purposes and for purposes of use in the mouth.

Compositions using mineral salts with an oily based carrier are known. They however have disadvantages when used as a scrub or exfoliating cleansing composition. Such scrub and exfoliating compositions would be particularly useful on human skin and tissue.

A need exists for a new cosmetic, therapeutic and tissue cleansing and moisturizing composition.

### SUMMARY OF THE DISCLOSURE

According to a first aspect, a chemical composition is disclosed, the composition comprising Dead Sea Mineral Salts (DSMS), Tea Trea Extract (TTE), and Urea in a suitable carrier or vehicle.

According to a second aspect, a composition is disclosed, which comprises: water and, emulsified and dispersed in the water, minerals salts, preferably DSMS, TTE and Urea.

According to a third aspect, a composition is disclosed which includes the use of salts, preferably mineral salts and more preferably, DSMS in conjunction with other components, such as a lotion, to create a cleansing composition for use as an exfoliating and scrubbing composition for the body or face tissue of a human or animal. Preferably the composition includes also TTE and/or Urea.

According to a fourth aspect, a composition is disclosed that is prepared with a water soluble and moisturizing lotion which retains the effective action of the DSMS. The DSMS are preferably included as granules, the granules normally being water soluble. TTE and/or Urea are also preferably included in the composition.

According to a fifth aspect a process is described, the process being for the manufacturing of a chemical composition, the process comprising combining DSMS, TTE and Urea.

According to a sixth aspect, a method to treat a condition of an epithelial tissue in human or animal is disclosed, the condition associated with a pathology, disease or disorder of the human or animal. The method comprises applying to the epithelial tissue a composition comprising DSMS, TTE and/or urea.

These and other features, aspects, and advantages of the present disclosure will become better understood with reference to the following description and appended claims.

### DETAILED DESCRIPTION OF THE PREFERRED EMBODIMENT

The compositions of the disclosure incorporate a combination of the ingredients particularly designed to provide a cosmetic or therapeutic effect.

DSMS refers to a mineral salts based composition obtainable by fractional evaporation and crystallization of salts from the Dead Sea brines, or active portions thereof. According to a first embodiment DSMS include Magnesium Chloride, Potassium Chloride and Sodium Chloride. According to a second embodiment the DSMS further include Calcium Chloride, Magnesium Bromide, Sulphates, and insolubles. Each of the above mineral salts can be included in the composition of the disclosure at any of the concentrations of the salt present in the composition obtainable from Dead Sea brines as described above. As the procedure to perform fractional evaporation and crystallization of salts from sea brines are known to a person skilled in the art, it does not need to be described here in detail.

TTE refers to an extract obtainable from a tea trea plant by procedure known in the art. Preferably, tea trea extract is an oil obtainable by crushing and distilling the leaves of a tea trea plant. The tea trea plant is preferably Melaleuca alternifolia.

Urea indicates the compound currently also known as carboxamide. Urea can be manufactured from ammonia and carbon dioxide or otherwise derived following procedures known to the person skilled in the art.

As suitable carrier or vehicle for the composition herein described are identifiable by the person skilled in the art, they do not need to be described here in detail.

Preferred combinations of the ingredients in cosmetic compositions include a DSMS amount from 2% to 7%, a TTE amount from 0.1%-0.5% and a Urea amount from 5% to 15%.

Preferred combinations of the ingredients in therapeutic composition comprise a range of Urea in the DSMS from 2 tenth's of an ounce Urea to 16 ounces DSMS, up to 7% Urea 93% DSMS.

In a preferred embodiment, the proper ratio among DSMS and Urea is 1 to 3 tenth's of an ounce urea to 16 ounces of the DSMS. This ratio will still produce a free flowing mixture of the salt. Higher concentration of urea can be used without reducing the effect of the urea and of the DSMS.

The combination of ingredients in the cosmetic or therapeutic composition can be formulated for use with epithelial tissues, such as human or animal skin. In the latter case, formulations can be provided to obviate the effect of modern environmental conditions on the human and animal body, for example to provide protection from environmental pollution and to provide moisturization of the skin.

The composition including the combination of ingredients herein disclosed provides for tissue repair, protection and relief for epithelial conditions in humans or animals. In particular the composition provides for treatment of epithelial condition associated with pathologies, disorders or diseases such as psoriasis or diabetes. In addition to the tissue repair effect, the composition can simultaneously provide moisturization and control of oil when necessary or desirable.

In a preferred form of the disclosure, each of the compositions include a moisturizing and/or non-oily base element for retaining the DSMS granules in their granule form until used so that the mixture will act as an exfoliating scrub.

The non-oily base is water soluble and thereby permits easy removal without leaving an oily film on the skin. The effect however is to provide for moisturizing the skin.

Other scrubbing agents such as silica or pumice can selectively be the effective scrubbing agent or can be added to any of the compositions. The product can contain other scrubbing agents such as loofah or nut shells. An antibacteria agent can also be incorporated in each of the formulas of the disclosure.

A preferred composition herein disclosed includes a lotion which includes D.I. water, Methylparaben, Propylene glycol, Glycerin 96%, Steareth-2 (Lipocol S-2), Steareth-20 (Lipocol S-20), Cetyl Alcohol (Lipocol C), Stearyl Alcohol (Lipocol S), Safflower oil, Isopropyl myristate (Liponate IPM), DC 200 Fluid 100 cst., Propylparaben, Germail II and Fragrance at a low ratio, approximately 1:10, or one part lotion to about ten parts, there is added DSMS, TTE and Urea. Preferably, a minimum active amount, preferably the least amount of water is used in the formulation of the composition so that as part of the lotion the salt granules are not inclined break down.

Other cosmetic or therapeutic formulations, such as cleansing and mouth formulations can be provided in accordance with the present disclosure, wherein the amount of active components included is such that a cosmetic or therapeutic effect is achieved. A person skilled in the art can determine the relevant amounts based on the content of the present disclosure.

Some of these compositions include formulations suitable for entry into the mouth either as a dental toothpaste, cream or as a mouthwash, ointments or other formulations suitable to be applied to epithelial tissues of humans or animals.

Other aspects of the disclosure include using the mineral bath salts with therapeutic herbs. Preferably, the Masada dead sea mineral bath salts are used with organic healing herbs. Aromatic essences can be added to these compositions. These compositions can relieve or assist in alleviating many common ailments.

The ingredients (herein also indicated as components) of a preferred embodiment of the present disclosure, their proportions, and the sequences of which they are a part for the purposes of mixing are described.

A new combination of ingredients results in a skin composition that provides protection against many types of skin damage, particularly itching and psoriasis, as well as other types of skin damage that can occur. Such other conditions can be flaking, redness, eczema, rashes or dry skin. Moreover, the composition is useful for cleansing the skin tissue, hands and the body and can be an effective scrubbing and exfoliating composition.

The composition of the present disclosure comprises a base in which active ingredients or components are mineral salts, preferably DSMS, TTE and/or Urea. Other additives or ancillary components can be included. The additives or ancillary components may provide for further effects in the composition. For example an additive which is useful against itching is eugenol extract, namely an extract of cloves, and this can be added to the composition as required or desired.

Typically, the composition of the present disclosure further comprises ancillary components such as: (1) a lipid-soluble component; (2) an emulsifier component; (3) an antioxidant component; (4) a preservative component; (5) a solvent component; (6) a thickener component; (7) a hydrophilic component; and (8) fragrance. Exemplary ingredients included within these components are described in detail below with reference to a preferred skin cream composition.

The ingredients are dispersed in an emulsified composition by the method of preparation described below. “Dispersed” refers to any process by which the ingredients are uniformly distributed in the emulsified base, and includes dissolving, emulsifying, and forming a colloidal suspension.

In a preferred skin cream composition the following ingredients are included in the indicated proportions

A. The Active Components

An essential ingredient of the disclosure are mineral salts such as Dead Sea salts. These Dead Sea salts are salts obtained from the region of Israel or Jordan known as the Dead Sea, by fractional evaporation and crystallization of salts, and generally include Magnesium Chloride, Potassium Chloride and Sodium Chloride together with other salts such as Calcium Chloride, Magnesium Bromide and Sulphides.

In a preferred embodiment DSMS used in the composition comprise at least the following active ingredients:

| | | |
|---|---|---|
| Magnesium Chloride | (MgCl₂) | 31.0-35.0% |
| Potassium Chloride | (KCl) | 24.0-26.0% |
| Sodium Chloride | (NaCl) | 4.0-8.0% |
| Calcium Chloride | (CaCl₂) | 0.4-0.6% |
| Magnesium Bromide | (MgBr₂) | 0.3-0.6% |
| Sulphates | (SO₄) | 0.05-0.2% |

| | |
|---|---|
| Insolubles | 0.05-0.3% |
| Water of Crystallization | 34.0-38.0% |

The DSMS in the composition provide the effect of stimulating circulation, detoxifying, hydrating and softening dry or cracked skin.

Another essential component is TTE. TTE provides antibacterial and antifungal protection from infection. In a preferred embodiment, the TTE is an oil which includes terpinen-4-ol and 1.8 cineole at relative concentrations which are in compliance with national and international standards such as, for example, the Australian standard AS2782-1985.

An additional essential components is Urea. Urea enhances the skin's natural exfoliation process and improves moisture retention. Since Urea is natural part of skin, individual who suffer for of a very dry skin, often present an imbalance of natural urea component needed to maintain healthy hydrated skin.

Each of the active components, particularly the DSMS, contributes to the improved properties of the cosmetic and cleansing composition of the present disclosure is present in a quantity sufficient to increase the smoothness, decrease the lumpiness, or decrease the itchiness, or edema of skin to which the composition is applied. Moreover, it reduces itching and repairs skin, and act as a cleansing agent.

B. The Ancillary Components

The composition of the present disclosure can further comprise a lipid-soluble component to provide added smoothness. The lipid-soluble component can comprise at least one ingredient selected from the group consisting of: (1) dimethicone; (2) bisabolol; (3) polyoxyethylene fatty acid esters; (4) cetyl alcohol; (5) a glyceryl triester of a medium-chain carboxylic acid selected from the group consisting of tricaproin, tricaprylin, tricaprin, and mixtures thereof; (6) white petrolatum; and (7) mineral oil.

The ancillary components, whose use is optional but preferable, impart additional desirable properties to the skin cream composition of the present disclosure. These components can include: (1) a lipid-soluble component; (2) an emulsifier component; (3) an antioxidant component; (4) a preservative component; (5) a solvent component; (6) a thickener component; (7) a hydrophilic component; and (8) fragrance. Preferably, the composition of the present disclosure comprises all the ancillary components as indicated below:

As comprised in a skin cream, the composition with the solvent provides for greater uniformity and ease of preparation. The solvent component can include one or two ingredients.

The thickener component improves the flow and rheological properties of the cosmetic composition. It permits the composition to be retained in a formulated state when applied to the skin. One or more ingredients can comprise the thickener component. The hydrophilic component can comprise one or multiple ingredients.

If desirable, a preservative component can be used to retard microbial and mold growth in the composition. The preservative component can also act as a stabilizer. The lipid-soluble component can also comprise one or multiple ingredients, and these can be varied depending upon the cosmetic composition as to be used. For instance, the composition can be formulated for normal skin, dry skin, or oily skin, and different lipid components can be used under those conditions.

1. The Lipid-Soluble Component

The cosmetic composition can comprise the additional ancillary ingredients whose use is optional but preferable. These ancillary ingredients can include a solvent component, a preservative component, a thickener component, a hydrophilic component, a lipid-soluble component and a pigment. As necessary or suitable, a fragrant component can also be added. Various combinations of these ingredients can be used as the solvent, lipid, thickener or hydrophilic component.

The glyceryl triester of the medium-chain carboxylic acid can be tricaprylin. The lipid-soluble component comprises dimethicone, bisabolol, polyoxyethylene fatty acid esters, cetyl alcohol, tricaprylin, white petrolatum, and mineral oil.

The dimethicone can comprise about 0.75% of the composition, the bisabolol comprises about 0.1% of the composition, the polyoxyethylene fatty acid esters comprise about 2.5% of the composition, the cetyl alcohol comprises about 4% of the composition, the tricaprylin comprises about 7.5% of the composition, the white petrolatum comprises about 3% of the composition, and the mineral oil comprises about 6.0% of the composition.

2. The Emulsifier Component

The skin cream composition of the present disclosure can further comprise an emulsifier component. Emulsifiers serve two functions. They act like a solubilizing agent to combine the water-soluble and non-water-soluble phases together; that is, to form a stable bridge between the waters and the oils of the ingredients. The emulsifiers also serve as emollients, providing a pleasant, esthetically appropriate tactile feeling when the emulsified composition is applied to the skin. The emulsifier component is present in a quantity sufficient to combine water-soluble and non-water-soluble phases of the composition.

The emulsifier component can comprise at least one of a mixture of mono- and distearate esters of polyoxyethylene and free polyethylene oxide, partial esters of lauric, palmitic, stearic, and oleic acids and hexitol anhydrides, and 120-mole ethoxylated jojoba oil. The emulsifier component can comprise a mixture of mono- and distearate esters of polyoxyethylene and free polyethylene oxide, partial esters of lauric, palmitic, stearic, and oleic acids and hexitol anhydrides and 120-mole ethoxylated jojoba oil.

Preferably, the mixture of mono- and distearate esters of polyoxyethylene and free polyethylene oxide comprises about 1.25% of the composition, the partial esters of lauric, palmitic, stearic, and oleic acids and hexitol anhydrides comprise about 0.13% of the composition, and the 120-mole ethoxylated jojoba oil comprises about 1.0% of the composition.

3. The Antioxidant Component

The composition according to the present disclosure can further comprise an antioxidant component. The antioxidant component prevents oxidation of the ingredients of the composition. The antioxidant component can be a mixture of 70% propylene glycol, 20% prowl gallate, and 10% citric acid. The antioxidant component comprises about 0.0011% of the composition.

4. The Preservative Component

The skin cream composition according to the present disclosure can further comprise a preservative component. The preservative component is used to prevent the growth of microbes in the emulsified skin cream composition, which is typically manufactured under clean, but non-sterile conditions. The preservative component can comprise at least one of imidazolyl urea and a complex of propylene glycol, phenoxyethanol, chlorphenesin and methylparaben.

The preservative component can comprises both imidazolyl urea and a complex of propylene glycol, phenoxyethanol, chlorphenesin and methylparaben, the imidazolyl urea comprises about 0.0011% of the composition, and the complex of propylene glycol, phenoxyethanol, chlorphenesin and methylparaben comprises about 2.5% of the composition.

In the complex of propylene glycol, phenoxyethanol, chlorphenesin and methylparaben, the propylene glycol comprises from about 30% to about 45% of the complex, the phenoxyethanol comprises from about 22% to about 37% of the complex, the chlorphenesin comprises from about 11% to about 22% of the complex, and the methylparaben comprises from about 11% to about 22% of the complex.

5. The Solvent Component

The composition according to the present disclosure can further comprise a solvent component. The use of a solvent component allows greater uniformity and ease of preparation. The solvent component can include at least one ingredient selected from the group consisting of ethylene glycol, propylene glycol, and 1.3-butylene glycol. The solvent component can comprise 1.3-butylene glycol and the solvent component comprises about 40% of the composition.

6. The Thickener Component

The composition according to the present disclosure can comprise a thickener component in a quantity sufficient to retain the composition when it is applied to the skin of a wearer. The thickener component can comprise at least one ingredient selected from the group consisting of alginate derivatives and preneutralized carbomer 430. The skin cream composition can comprise both alginate derivatives and preneutralized carbomer 430. The alginate derivative can comprise about 0.2% of the composition, and the preneutralized carbomer 430 and be about 0.5% of the composition.

7. The Hydrophilic Component

A composition according to the present disclosure can further comprise a hydrophilic component. The hydrophilic component can comprise a polar complex consisting essentially of mannitol, arginine, serine, pyrrolidone carboxylate, sucrose, citrulline, glycogen, histidine, alanine, threonine, glutamic acid, and lysine. The polar complex can comprise about 1.0% of the composition.

8. Fragrance

The composition according to the present disclosure can further comprise fragrance. The use of fragrance is well known in the cosmetic art, and need not be described further. The fragrance can comprise about 0.45% of the composition, although this can vary depending upon the fragrance used.

Compositions according to the present disclosure can further comprise other components used in the cosmetic art, such as pigments and other conventional excipients. The use of such ingredients is well known in the cosmetic and cleansing art and need not be described further here. The pigment component gives the cosmetic composition an aesthetically desirable appearance and different pigments can be used as variables in relation to the skin tone of the intended user.

Other components of the composition which permit for its application to the skin include propylene, glycol, and effective amounts of aloe vera juice, sunflower oil, kessco OP, liponate, other hydrogenated vegetable oil, lipo GMS, stearic acid, and lipomulse. As necessary, the product could also include, when intended for application to the skin, a suitable sunscreen composition. In different forms, although aloe vera juice is considered, other extracts are possible. This could, for instance, be extracts from other fruits such as balsalm or apricot.

An example of the cosmetic composition is set out as follows, together with the relative percentages of content, and also an indication of the relative phase of those products. The relationship of the phase is also set out in regard to the method of formulating the composition.

### EXAMPLE 1

### Sample Composition for Cosmetic Cleansing and Moisturizing Product

The following is a table reporting the components of a sample composition according to the disclosure.

| PHASE | % | MATERIAL |
|---|---|---|
| 1 | 64.9500 | DEIONIZED WATER |
| | 1.000 | ALOE VERA JUICE |
| | 0.2000 | METHYLPARABEN |
| 2 | 3.0000 | PROPYLENE GLYCOL |
| | 0.1000 | KELTROLT |
| 3 | 1.0000 | SUNFLOWER OIL |
| | 2.0000 | KESSCO OP |
| | 2.0000 | LIPONATE GC |
| | 1.0000 | THODORSIL 47V 350/SF 96-350 |
| | 2.0000 | HYDROGENATED VEGETABLE OIL |
| | 3.5000 | LIPO GMS 470 - WITCONOL 2407 |
| | 2.0000 | STEARIC ACID |
| | 2.0000 | LIPOMULSE 165/ARLACEL 165V |
| | 1.0000 | LIPOCOL C/ALFOL 16 |
| | 1.0000 | LIPO WAX D |
| | 0.1000 | PROPYLPARABEN |
| | 0.1000 | VITAMIN E ACETATE |
| 4 | 0.5000 | EUGENOL EXTRA USP |
| | 0.4000 | MACKSTAT DM |
| | 0.5000 | WHITE DIAMOND B/M63149 |
| | 0.1000 | COVIOX T70 |
| 5 | 10.0000 | DEIONIZED WATER |
| | 5.0000 | DEAD SEA SALT |
| TOTAL | 103.0000 | |

The method of formulation of such a sample is as follows:

| STEP | TEMP | |
|---|---|---|
| 1 | 80° | PHASE 1 |
| | | Into a main SS compounding tank, meter Deionized |
| | | Water. Add Aloe Vera. Turn on mixer. Begin |
| | | heating to 80° C.. |
| 2 | 70° | When the temperature of the main tank is at |
| | | 70° C. sprinkle slowly Methylparaben. |
| | | Mix well until completely dissolved. |
| | | Recirculate batch to insure that no solid |
| | | is left at the bottom. |
| 3 | RT | PHASE 2 |
| | | Into a suitably size container, add Phase 2 |
| | | ingredients. Mix well into a uniform slurry. |
| 4 | 80° | When the temperature of the main tank is at |
| | | 80° C. add slowly Phase 2 slurry. Note: |
| | | Be sure to mix Phase 2 slurry well before |
| | | adding to Phase 1. Keltrol T has a tendency |
| | | to settle at the bottom. |
| 5 | 80° | PHASE 3 |
| | | Into a separate ss auxiliary tank, add Phase 3 |
| | | ingredient except Propylparaben. Begin heating |
| | | to 80° C.X. Turn on mixer when most |
| | | of the waxes are melted. |
| 6 | 70° | When temperature of Phase 3 is at 70° C. |
| | | sprinkle in slowly Propylparaben. Be sure to |
| | | evacuate valve to insure that no solids |
| | | are entrapped. |
| 7 | 80° | When the temperature of Phase 3 is at 80° C. |
| | | and ready to be added to Phase 1, add Vitamin E |
| | | Acetate. |
| 8 | 80° | EMULSIFICATION |
| | | When both Phase 1 and Phase 3 reach 80° C., |
| | | add Phase 3 to main batch. Mix well for |
| | | 15 minutes. Recirculate batch on and off. |
| 9 | | Begin cooling. |
| 10 | 38° | PHASE 4 |
| | | When temperature of the main batch is at |
| | | 38° C. add Phase 4 ingredients. Mix well. |
| 11 | RT | PHASE 5 |
| | | Into a suitably sized container add Phase 5 |
| | | ingredients. Mix well until completely dissolved. |
| 12 | 38° | When the temperature of the main batch is 38° |
| | | add the solution of Phase 5. Mix well. |
| 13 | 30° | Discontinue mixing and cooling at 30° C. |

TTE and Urea may also be added to a final concentration which is in accordance with DSMS/TTE/Urea ratios and relative percentages indicated in the present disclosure.

### EXAMPLE 2

### Sample Scrubbing or Exfoliating Composition

Such a composition would include the following ingredients:

| MATERIAL | % |
|---|---|
| D.I. water | 75.28 |
| Methylparaben | 0.25 |
| Propylene glycol | 2.00 |
| Glycerin 96% | 5.00 |
| Steareth 2 (Lipocol S-2) | 1.00 |
| Steareth-20 (Lipocol S-20) | 2.00 |
| Cetyl Alcohol (Lipocol C) | 1.50 |
| Stearyl alcohol (Lipocol 5) | 1.50 |
| Safflower oil | 4.00 |
| Isopropyl myristate (Liponate IPM) | 5.00 |
| DC 200 Fluid II cst. | 1.00 |
| Propylparaben | 0.07 |
| Germail II | 0.20 |
| Fragrance | 0.20 |

Additionally, there is Dead Sea salts added in a range of about 1:10, namely ten parts salt to one part of lotion. As needed, other scrubbing agents such as pumice or silica can be added.

TTE and Urea may also be added to a final concentration which is in accordance with DSMS/TTE/Urea ratios and relative percentages indicated in the present disclosure.

Although the disclosure has been described with regard to a cosmetic product related to soothing the effects of psoriasis, there are other formulations of the product which essentially includes mineral salts, preferably Dead Sea salt which can be used for different purposes. As such, a mouthwash formulation can be provided. The product will assist in combatting bacteria and gum irritation and inflammation. The product can moisturize and assist in nourishment of the skin.

The Dead Sea salts can be formulated with an amount of water and a suitable fragrance for these purposes Likewise, as a shampoo product. the forrmulation can be prepared for use on human hair as well as animal hair An essential ingredient of such a product is also mineral salts, such as Dead Sea mineral salts, and the percentage of Dead Sea salt can vary according to the purpose and formulation.

### EXAMPLE 3

### Conditioning Shampoo Composition

The following is a table reporting the components of a sample composition according to the disclosure.

| TSP | % |
|---|---|
| Masada salts | 2.00 |
| DI water | 57.50 |
| Cocomido propyl betaine/(Norfox) | 7.00 |
| SLES-2/(Nortco)(Sodium laureth sulfate) | 20.00 |
| SLES-60/(Norfco) | 5.00 |
| Cocomido propyl amine codde/Schercamox CAA-G (Scher) | 2.50 |
| Crodafos SG (Croda) (PTG-S Ceteth-10 phosphate) | 1.00 |
| Oleamide DEA/Schercomid ODA (Scher) | 2.50 |
| Tritisol/sol wheat protein (Croda) (Hydrolyzed wheat protein) | 0.70 |
| Aloe 10:1 (D-D Chem) | 0.20 |
| Neutral henna | 0.10 |
| Algae extract | 0.10 |
| Comfrey leaf | 0.10 |
| Goldenscal root | 0.10 |
| Elderflower | 0.10 |
| Nettle | 0.10 |
| Rosemary | 0.10 |
| Red clover | 0.10 |
| panthenol (Roche) | 0.10 |
| DMDM hydantoin | 0.40 |
| Fragrance (Custom Essence) CE-22601 coal tar mask | 0.30 |
| TOTAL | 100.00 |

The procedure for mixing the formulation is as follows:

1. Add ingredients of phase A individually with mixing Mix until homogenous.

2. When phase A is homogeneous, add individually ingredients of phase B. Note: If scale up testing indicates that the fragrance is clouding the product, premix 1:1 with Triton X-100.

3. Adjust pH to around 6.

TTE and Urea may also be added to a final concentration which is in accordance with DSMS/TTE/Urea ratios and relative percentages indicated in the present disclosure.

Further, the different compositions and formulations can also be used, for instance, in the soothing of the skin when subjected to acne.

The composition is applied to the skin to stop itching. When applied to animals particularly it is useful against skin disorders, and joint or arthritic problems. It is particularly useful on dogs and horses.

Other forms of the disclosure includes those when applied in liquid form. It can also be applied as saturated wipe or as a compress. Moreover, it is applicable as a scrubbing or exfoliating composition. Additionally, it can be formulated as an inhalant product where another component is eucalyptus.

A different composition of the formula can be used for inhaling to ease nasal or sinus congestion and to soothe coughing irritations due to bronchitis or similar conditions.

### EXAMPLE 4

### Therapeutic Compositions

Masada dead sea mineral bath salts can be used with organic healing herbs and, selectively, aromatic essences. These compositions can be used to stimulate circulation, relieve aches and pains, soften skin, promote relaxation, and draw out toxins from the body. With the salts fortified with the organic herbs and aromatic essences, many common ailments can be at least partly relieved. Different compositions are set out as follows:

### EXAMPLE 4

### Therapeutic Compositions (Continued)

MASADA MINERAL HERB SPA BATH SALTS TREATMENT COLLECTION

| Therapy for | Therapeutic Organic Herbs | Aromatic Essences |
|---|---|---|
| PMS RELIEF | Chasteberry & Dandelion Root | Lavender & Geranium |
| | Helps relieve aches, cramping: | Relaxes and calms sensitivities |
| | Eases excess water retention | Helps lift blue moods |
| | discomfort | |
| COLDS & FLU | Echinacea, Goldenseal Root, | Eucalyptus |
| | Lemon Balm | Helps relieve sinus congestion |
| | Helps strengthen the body's | Reduces inflammation |
| | immune system | |
| | Eases flu aches and pains | |
| DETOX, M.D. | Green Tea, Milk Thistle Root, | Rosemary & Juniper |
| | Ginseng Root, Rosemary & | Combats mental fatigue |
| | Juniper | Enhances physical energy |
| | Invigorates and fortifies the body | |
| | Draws out toxins, stimulating | |
| | circulation | |
| TOTAL RELAXATION | Valerian, Kava Kava & Passion | Lavender, Orange Blossom and |
| | Flower Calms frayed nerves and | Vetivyr |
| | helps promote sleep | Relaxes the mind & body |
| | Helps relieve tension and | Promotes peace & balance |
| | fatigue Relaxes tense, aching | |
| | muscles | |
| SKIN CALMING | Calendula, Clove & Chamomile | Chamomile & Melissa & Linden |
| | Excellent for psoriasis and | Soothes irritation and redness |
| | eczema | Eases stress and tension |
| | Relieves itchy. burning. irritated | |
| | skin Eases discomfort from | |
| | allergies. insect bites | |
| | Clams and soothes swelling | |
| JOINT & MUSCLE | Thyme, Juniper Berries & | Wintergreen |
| RELIEF | Yarrow Winter-green. Camphor | Warms stiff muscles and joints |
| | & Rosemary | Eases tension |
| | Excellent for arthritis and | |
| | rheumatism | |
| | Relaxes muscles and soothes | |
| | cramps and aching joints | |

TTE and Urea may also be added to a final concentration which is in accordance with DSMSITE/Urea ratios and relative percentages indicated in the present disclosure.

Although the selected organic herbs are set out in the examples, it is clear that many other herbs can be used in a manner commonly used for different ailments and conditions. The herbs may also be non-organically produced or grown herbs. These conditions can be applicable both for humans and for animals, and the therapeutic uses can be for humans and for animals. An advantage of this technique is that where a patient cannot normally take herbs orally, the herbs can be absorbed through the skin by these bath salts products which can be used for bathing or showering and the like.

Other exemplary compositions according to the disclosure include different combinations of DSMS, TTE and Urea together with other components such as Botanical Green Tea, Clove, and Aloe Vera to provide anti-inflammatory and antioxidant protection and Vitamins A, C, E, and K to nourish and encourage natural skin renewal.

### EXAMPLE 5

### Antibacterial Dead Sea Mineral Therapy

This composition can be used for skin therapy of diabetes associated skin conditions. The components are included in new DiabEase® skin care products in a combination that helps relieving skin conditions related to diabetes and provides a total self-management skin protection regimen for daily health maintenance.

The above composition can be formulated to include any combination of the ingredients according to the disclosure. Preferably this composition can include any combination ingredients from 2 tenth's of an ounce Urea to 16 ounces DSMS, up to 7% Urea 93% DSMS.

### EXAMPLE 6

### Dead Sea Mineral Bath Salts with Antibacterial Tea Tree and Hydrating Urea

This composition is exclusively formulated for diabetic skin care and includes DSMS with antibacterial Tea Tree and deeply hydrating Urea. This potent ingredient complex includes the components at a concentration that goes beyond the benefits of Dead Sea Mineral Bath Salts to provide enhanced moisturizing for dry, cracked and irritated skin. Soaking in these salts stimulates circulation, helps detoxify the body and supports its natural bacteria fighting process, while restoring the skin's natural moisture balance.

The above composition can be formulated to include any combination of the ingredients according to the disclosure. Preferably this composition can include any combination ingredients from 2 tenth's of an ounce Urea to 16 ounces DSMS, up to 7% Urea 93% DSMS.

### EXAMPLE 7

### Hydrating Skin Therapy with Antibacterial Tea Tree, Hydrating Urea and Aloe Vera

A lightweight, and richly-hydrating cream that can be used for a moisturizing treatment to protect and nourish the skin and includes DSMS Aloe Vera, Allantoin, and Vitamins A and E as antioxidants and protect the skin from environmental stress that can cause dryness and irritation. Urea is included in amounts and proportion makes skin ultra soft and hydrated. Antibacterial Dead Sea Minerals and antiseptic Tea Tree Oil are included in amounts and proportion that helps protect the skin from impurities. Regular use helps prevent cracked skin and callused areas that may lead to infection.

The above composition can be formulated to include any combination of the ingredients according to the disclosure, in amounts and proportion that ensure the above effects. One composition include the following ingredients: Deionized Water, Urea, Sunflower Seed Oil, Propylene Glycol, Dead Sea Minerals, Glyceryl Stearate and PEG-100 Stearate, Stearic Acid, Safflower Oil, Cetyl Alcohol, Stearyl Alcohol, Melaleuca Alterifolia (Tea Tree) Leaf Oil, Sorbitan Isostearate, Triethanolamine, Aloe Vera Extract, Allantoin, Tocopheryl Acetate (Vitamin E), Retinyl Palmitate (Vitamin A), Octyl Palmitate, Cholecalciferol (Vitamin D), Camillia Sinensis (Green Tea) Extract, Methylparaben, Propylparaben, Imidazolidinyl Urea, Fragrance.

### EXAMPLE 8

### Skin Calming Cream with Antibacterial Tea Tree, Soothing Clove and Hydrating Urea

This is a therapeutic soothing cream that quiets itching and irritation so common in diabetic skin. While comforting the skin, DSMS in therapeutic concentrations and proportions such that they penetrate deeply the skin to detoxify, stimulate circulation, promote the natural healing process and/or preventing future irritation. Antioxidant Green Tea, nourishing Vitamins, soothing Oat and anti-inflammatory Clove Oil are also included in amounts and proportion that help protect skin and/or restore the skin to its health condition. Some formulations also contains Eugenol from Clove Oil in amounts and proportions that help quickly quiet irritation.

The above composition can be formulated to include any combination of the ingredients according to the disclosure, in amounts and proportion that ensure the above effects. One composition includes the following ingredients: Water, Dead Sea Mineral Salt, Glyceryl Stearate, Propylene Glycol, Ethyihexyl Palmitate, Caprylic/Capric Triglyceride, Hydrogenated Vegetable Oil, Stearic Acid, Aloe Barbadensis, Carthamus Tinctorius (Safflower) Seed Oil, Dimethicone, Cetyl Alcohol, Cetearyl Alcohol, Ceteareth-20, Urea, Eugenol, Avena Sativa (Oat) Extract, Phytonadione (vitamin k), Retinyl Palmitate (vitamin a), Ascorbic Acid (vitamin c), Chamomilla Recutita Extract, Camillia Sinensis (Green Tea) Extract, Melaleuca Alternifolia (tea tree) Leaf Oil, Tocopheryl Acetate (vitamin F), Xanthan Gum, Tocopherol (vitamin E), Methylparaben, Ethylparaben, Phenoxyethanol.

### EXAMPLE 9

### Callus Therapy Cream with Antibacterial Tea Tree and Softening Urea

A composition that can be used in a gentle, intensive, hydrating and softening treatment helps remove calluses without irritating the skin. Dead Sea Minerals with antibacterial Tea Tree are included in amounts and proportions that protect and purify skin. Urea is included in amounts and proportions that both exfoliates rough surface layers and brings moisture deep into the skin to soften and soothe, in particular into dry, cracked, and hardened areas on feet, elbows, hands and fingers.

The above composition can be formulated to include any combination of the ingredients according to the disclosure, in amounts and proportion that ensure the above effects. One composition is formulated to include the following Ingredients: Deionized Water, Sunflower Seed Oil, Urea, Dead Sea Minerals, Stearic Acid, Glyceryl Stearate and PEG-100 Stearate, Propylene Glycol, Safflower Oil, Aloe Vera Gel, Cetyl Alcohol, Melaleuca Alterifolia (Tea Tree) Leaf Oil, Potassium ilydroxide, Sorbitan Isostearate, Triethanolamine, Dimethicone, Allantoin, Tocopheryl Acetate (Vitamin E), Retinyl Palmitate (Vitamin A), Camillia Sinensis (Green Tea) Extract, Methylparaben, Propylparaben, Imidazolidinyl Urea, Aromatic Essences.

### EXAMPLE 10

### Foot Therapy Salts with Antibacterial Tea Tree and Hydrating Urea

Masada Dead Sea Mineral Salts are infused with antibacterial Tea Tree and hydrating Urea in amounts and proportion that provide purifying and softening benefits. In particular DSMS Teat Tree and Urea are included in amounts and proportion that soften dry, cracked skin and stimulates circulation to encourage natural renewal. Regular soaking in this composition helps preventing calluses and infections.

The above composition can be formulated to include any combination of the ingredients according to the disclosure, in amounts and proportion that ensure the above effects.

### EXAMPLE 11

### Foot Therapy Scrub with Antibacterial Tea Tree and Vitamins C & K

A gently exfoliating scrub with antibacterial Tea Tree and Vitamins C and K in amounts and proportion that purify and softens the feet, lifting away rough, dry, cracked skin. A regular exfoliation with this scrub helps preventing calluses and keep feet healthy. In particular, Tea Tree helps is included in amounts and proportions that deep clean the feet and protect skin from impurities. Vitamin C is included in amounts and proportions that smooth the surface texture; Vitamin K is included in amounts and proportions that have fortifying effect on the skin; antioxidant Green Tea and toning Thyme, Sage, and Rosemary are included in amounts and proportions help revitalize the skin. Regular exfoliation stimulates circulation, helps keep the skin soft and smooth, and may help prevent the formation of cracked, callused skin.

The above composition can be formulated to include any combination of the ingredients according to the disclosure, in amounts and proportion that ensure the above effects. One composition is formulated to include the following Ingredients: Water, Carthamus Tinctorius (Safflower) Seed Oil, Walnut Shell Powder, Stearic Acid, Glyceryl Stearate, Cetyl Alcohol, Glycerin, Dead Sea Mineral Salt, Propylene Glycol, PEG-100 Stearate, Menthol, Aloe Barbadensis, Pumice, Camillia Sinensis (Green Tea) extract, Panax Ginseng, Thymus Vulgaris (Thyme) Extract, Melaleuca Alternifolia (tea tree) Leaf Oil, Ascorbic Acid (vitamin c), Phytonadione (vitamin k), Salvia (sage) extract, Rosemarinus Officinalis (rosemary) extract, Citric acid, Hydroxyethylcellulose, Tetrasodium Edta, Phenoxyethanol, Tocopherol (vitamin E), Ethylparaben, Propylparaben, aromatic essences.

### EXAMPLE 12

### Foot Therapy Cream with Antibacterial Tea Tree and Hydrating Urea

This deeply moisturizing foot cream is formulated to provide a protective shield for the feet. Natural Dead Sea Minerals and Urea soften and hydrate dry, cracked skin while Vitamin C smoothes the surface texture and promotes natural healing. Wintergreen stimulates circulation and refreshes the feet.

The above composition can be formulated to include any combination of the ingredients according to the disclosure, in amounts and proportion that ensure the above effects. One composition is formulated to include the following ingredients: Water, Ethylhexyl Palmitate, Cetearyl Alcohol, Glyceryl Stearate, Glycerin, PEG-100 Stearate, Carthamus Tinctorius (Safflower) Seed Oil, Dimethicone, Hydrogenated Vegetable Oil, Dead Sea Mineral Salt, Elacis (iuineensis (Palm) Kernel Oil, Aloe Barbadensis, Methyl Salicylate (Wintergreen), Camillia Sinensis (Green Tea) Extract, Retinyl Palmitate, Phytonadione (vitamin k), Ascorbic Acid (vitamin c), Retinyl Palmitate (Vitamin A), Melaleuca Alternifolia (tea tree) Leaf Oil, Propylene Glycol, Hydroxyethylcellulose, Tocopherol (vitamin E), Polysorbate 60, Steareth-2, Tetrasodium edta, Methylparaben, Ethylparaben, Phenoxyethanol, Menthol, peppermint (Essential Oil).

The disclosure has been described with regard to specific embodiments and several examples which are for illustrative purposes only. Other embodiments and examples will be apparent to those of ordinary skill in the art in view of the foregoing description. The scope of protection of the present disclosure is defined by the appended claims.

We claim:

1. A composition comprising a mineral salt having as active ingredients Magnesium Chloride, Potassium Chloride, Sodium Chloride, tea tree oil, urea, water, a lipid soluble compound, oils, and a herbal component, the composition having components present in sufficient quantities to effectively minimize against a condition of at least one of bodily muscular cramp, excess water retention and swelling.

2. A composition according to claim 1, further comprising an aromatic essence.

3. A composition according to claim 1, comprising at least about 5% Dead Sea salts.

4. A composition according to claim 1, further comprising an organic herb component.

5. A composition according to claim 1, wherein said mineral salt is emulsified and dispersed in water.

6. A composition according to claim 4, wherein the herb component is selected to be at least one of chasteberry, dandelion root, echinacea, golden seal root, lemon balm, green tea, milk thistle root, ginseng root, rosemary, juniper, valerian, kava kava, passion flower, calendula, clove, chamomile, thyme, juniper berries, yarrow wintergreen, camphor or rosemary.

7. A composition according to claim 2, wherein the essence is at least one of lavender, geranium, eucalyptus, rosemary, juniper, lavender, orange blossom, vetivyr, chamomile, melissa, linden or wintergreen.

8. A composition according to claim 1, comprising a compound for application to the body as a bath salt during bathing or showering.

9. A composition comprising Dead Sea Mineral Salts, Tea Tree extract and urea in a suitable carrier or vehicle.
